(19) United States
(12) Patent Application Publication
(10) Pub. No.: US 2026/0145688 A1
(43) Pub. Date: May 28, 2026

(54) VEHICLE BASED ALLERGEN SENSITIVITY MONITORING

(71) Applicant: GM Global Technology Operations LLC, Detroit, MI (US)

(72) Inventors: Tetyana Wasko, West Bloomfield, MI (US); Russell A. Patenaude, Macomb Township, MI (US)

(21) Appl. No.: 18/959,978

(22) Filed: Nov. 26, 2024

(51) Int. Cl. B60W 50/00; B60W 40/02; B60W 40/08; G06V 20/59; G06V 40/10

(52) U.S. Cl. CPC B60W 50/0098; B60W 40/02; B60W 40/08; G06V 20/597; G06V 40/10; B60W 2040/0872; B60W 2420/403; B60W 2420/408; B60W 2420/54; B60W 2540/221; B60W 2555/20; B60W 2556/10

(57) ABSTRACT

A vehicle includes a controller having a processor and a memory. The controller has an allergen sensitivity detection module. A physiological sensor suite in the vehicle is configured to identify a physiological response of an occupant. The vehicle also includes an air quality sensor suite with air quality sensors and at least one camera defining a field of view including the occupant. The allergen sensitivity detection module includes instructions configured to cause the processor to detect a physiological response of the occupant, identify a correlation between the physiological response of the occupant and a detected allergen, generate a response to the identified correlation.

The subject disclosure relates to vehicles, and in particular to monitoring vehicle air quality based on detected occupant reactions.

Detecting and diagnosing airborne allergen sensitivity at home can be challenging for several reasons. Firstly, identifying specific allergens in the air requires specialized equipment and testing methods that are not be readily available to individuals at home. Additionally, symptoms of allergies can be subtle or mistaken for other conditions, leading to delayed recognition. Allergies can also develop gradually over time, due to individuals being exposed to allergens without realizing the connection to their symptoms. This delayed realization can result in years passing before someone recognizes they have an allergy and seeks proper diagnosis and treatment.

Some vehicles include air quality monitoring systems, however, existing air quality monitoring systems typically monitor and test environmental air quality as the air is being ingested into the vehicle. While this monitoring provides insight into the surrounding environment, the monitoring does not provide tailored information for each occupant of the vehicle. Nor does the monitoring track vehicles for contaminants internal to the passenger compartment of the vehicle such as pet dander, tracked in pollen, dust and the like.

Furthermore, while existing air quality monitoring systems provide static insight with regards to the air quality, the existing systems are not able to account for changes in the occupant. As a result, initial alert levels that may be set and/or allergen types that are identified by the occupant may not be accurate at a future point in time.

It is desirable to provide an allergen sensitivity monitoring system for a vehicle that accounts for allergens that are brought into the vehicle through routes other than an air intake and for a vehicle that monitors one or more occupants dynamic allergen sensitivity over time.

### SUMMARY

In one exemplary embodiment a vehicle includes a controller having a processor and a memory. The controller has an allergen sensitivity detection module. A physiological sensor suite in the vehicle is configured to identify a physiological response of an occupant. The vehicle also includes an air quality sensor suite with air quality sensors and at least one camera defining a field of view including the occupant. The allergen sensitivity detection module includes instructions configured to cause the processor to detect a physiological response of the occupant, identify a correlation between the physiological response of the occupant and a detected allergen, and generate a response to the identified correlation.

In addition to one or more of the features described herein the response generated includes at least one of outputting an allergen sensitivity report to the occupant, notifying a first responder, adjusting at least one in vehicle airflow parameter, rerouting a vehicle navigation system, and rerouting an autonomous driving system.

In addition to one or more of the features described herein the response generated is dependent on a severity of the physiological response.

In addition to one or more of the features described herein the identifying the correlation between the physiological response of the occupant and the detected allergen comprises scoring a severity of the physiological response on a physiological response scale, normalizing a detected magnitude of at least one allergen to the physiological response scale, and identifying a correlation between the physiological response and the at least one allergen in response to a physiological response score exceeding a threshold and the normalized magnitude of the at least one allergen exceed the threshold.

In addition to one or more of the features described herein identifying the correlation between the physiological response of the occupant and the detected allergen includes receiving at least one occupant entered physiological response and recalculating the physiological response including the occupant entered physiological response.

In addition to one or more of the features described herein the air quality sensor suite includes a first set of sensors configured to monitoring allergen quantities in air ingested to the vehicle from an exterior environment and a second set of sensors configured to monitor air recirculated from a passenger compartment of the vehicle.

In addition to one or more of the features described herein the allergen sensitivity detection module further includes instructions configured to cause the processor to update an occupant history file with the identified correlation and compare the identified correlation with historical correlations.

In addition to one or more of the features described herein the generated response includes an output identifying a difference between the identified correlation and the historical correlation.

In addition to one or more of the features described herein the physiological sensor suite includes biosensors, cameras, microphones, and lidar sensors.

In addition to one or more of the features described herein the allergen sensitivity detection module includes instructions configured to cause the processor to iterate identifying the correlation between the physiological response of the occupant and a detected allergen, generate a response to the identified correlation at a predetermined interval.

In addition to one or more of the features described herein the allergen sensitivity module includes instructions configured to cause the processor to continuously detect a physiological response of the occupant and monitor allergen levels.

In another exemplary embodiment a method for monitoring an allergen sensitivity of an occupant of a vehicle includes detecting a physiological response of the vehicle occupant using a physiological sensor suite of the vehicle. A magnitude of at least one allergen in the vehicle is detected using an air quality sensor suite of the vehicle. A correlation between the physiological response of the occupant and the detected magnitude of the at least one allergen is detected using a controller of the vehicle. A vehicle response to the identified correlation is implemented using the controller.

In addition to one or more of the features described herein the vehicle response includes at least one of outputting an allergen sensitivity report to the occupant, notifying a first responder, adjusting at least one in vehicle airflow parameter, rerouting a vehicle navigation system, and rerouting an autonomous driving system.

In addition to one or more of the features described herein the vehicle response is dependent on a severity of the physiological response.

In addition to one or more of the features described herein identifying the correlation between the physiological response of the occupant and the detected allergen comprises scoring a severity of the physiological response on a physiological response scale, normalizing a detected magnitude of at least one allergen to the physiological response scale, and identifying a correlation between the physiological response and the at least one allergen in response to a physiological response score exceeding a threshold and the normalized magnitude of the at least one allergen exceed the threshold.

In addition to one or more of the features described herein identifying the correlation between the physiological response of the occupant and the detected allergen includes receiving at least one occupant entered physiological response and recalculating the physiological response including the occupant entered physiological response.

In addition to one or more of the features described herein the air quality sensor suite includes a first set of sensors configured to monitoring allergen quantities in air ingested to the vehicle from an exterior environment and a second set of sensors configured to monitor air recirculated from a passenger compartment of the vehicle.

In addition to one or more of the features described herein the method includes updating an occupant history file with the identified correlation and compare the identified correlation with historical correlations.

In addition to one or more of the features described herein the vehicle response includes an output identifying a difference between the identified correlation and the historical correlation.

In addition to one or more of the features described herein, the method includes iterating identifying the correlation between the physiological response of the occupant and a detected allergen, generate a response to the identified correlation at a predetermined interval.

The above features and advantages, and other features and advantages of the disclosure are readily apparent from the following detailed description when taken in connection with the accompanying drawings.

### BRIEF DESCRIPTION OF THE DRAWINGS

Other features, advantages and details appear, by way of example only, in the following detailed description, the detailed description referring to the drawings in which:

FIG. 1 depicts a vehicle including a dynamic allergen sensitivity monitoring system; and

FIG. 2 illustrates a method of operating the dynamic allergen sensitivity monitoring system accounting for one or more specific vehicle occupants.

### DETAILED DESCRIPTION

The following description is merely exemplary in nature and is not intended to limit the present disclosure, its application or uses. It should be understood that throughout the drawings, corresponding reference numerals indicate like or corresponding parts and features. As used herein, the term module refers to processing circuitry that may include an application specific integrated circuit (ASIC), an electronic circuit, a processor (shared, dedicated, or group) and memory that executes one or more software or firmware programs, a combinational logic circuit, and/or other suitable components that provide the described functionality.

As used herein, the term controller refers to a control configuration including a dedicated system having a processor and a memory, a distributed control configuration including multiple systems in communication with each other and configured to cooperatively implement a control scheme, a general system having one or more processors and memories and including subroutines, modules, or programs for affecting a particular control scheme, or any similar control architecture configured to affect a desired control scheme.

As used herein, the term sensor suite refers to sensors, communication connections between the sensors and one or more control elements (such as a controller), and any corresponding physical structures for supporting the sensors at a desired location. A sensor suite may include sensors distributed about a vehicle in addition to sensors positioned in a singular central location. The communication connections may include direct and indirect communication connections and may include wired and wireless communication connections. Identification and discussion of specific sensor structures and sensor type within the sensor suite is exemplary in nature and is not exhaustive. As such, additional sensor types and structures beyond those expressly identified may be incorporated and utilized as part of the sensor suite in any conventional manner.

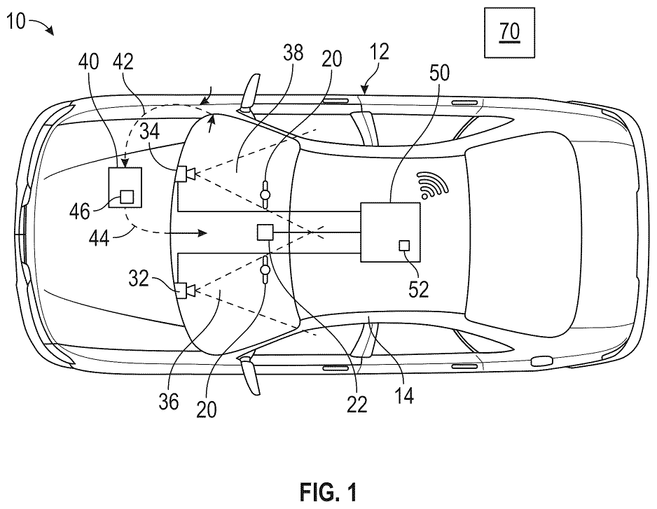

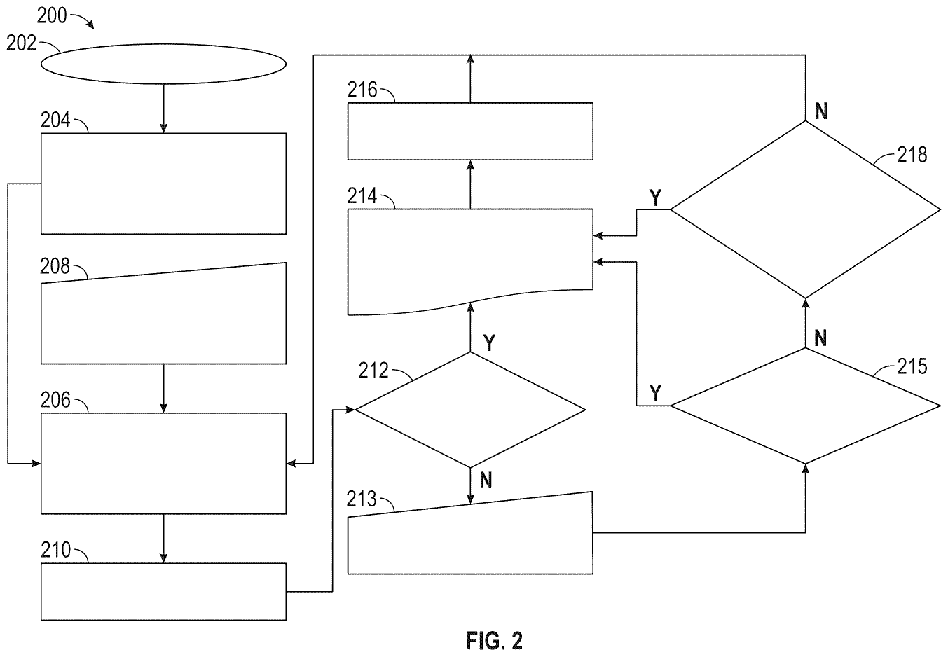

In accordance with an exemplary embodiment FIG. 1 illustrates a vehicle 10 including a vehicle body 12 and a passenger compartment 14. A driver and passenger (referred to collectively as occupant(s) 20) are positioned within the passenger compartment 14. The vehicle 10 further includes cameras 32, 34 each of which defines a corresponding field of view 36, 38 including one or more of the vehicle occupants 20. The cameras 32, 34 are in communication with a vehicle controller (controller 50). While illustrated at positions directly in front of each vehicle occupant 20, it is appreciated that the cameras 32, 34 may be positioned in alternate and/or additional locations throughout the passenger compartment 14. The controller 50 includes an allergen sensitivity detection module 52 for implementing an allergen sensitivity detection process 200 (illustrated in FIG. 2). The controller 50 further includes a memory storing occupant profiles which characterize data regarding uniquely identified occupants 20. The data can include allergy details generated and used by the allergen sensitivity detection module 52, as well as any other unique information about the uniquely identified occupant 20 such as climate preferences, driving habits, and the like.

A physiological sensor suite 22 monitors one or more physiological characteristics of the occupant(s) 20 and provides the monitored details to the controller 50. In some examples, the physiological sensor suite 22 includes biosensors, cameras, microphones, lidar and the like disposed in the passenger compartment 14 or otherwise configured to monitor occupants 20. The physiological sensors monitor the occupants 20 to detect any symptoms of allergic response and/or respiratory distress and provide the output data to the controller 50. Monitored symptoms may include, but are not limited to, abnormal breathing patterns, shortness of breath, excessive coughing or throat clearing, gasping or wheezing, sneezing frequency, and increased respiratory rate. In addition to respiratory parameters, image analysis and/or a combination of image analysis and other sensor feedback may be used by the controller 50 to identify watery or itchy eyes using camera 32, 34 images and gesture analysis for frequency, severity, length of rubbing, wiping, redness, etc). Additional physiological sensors may be used to analyze increased heart rate, decreased blood oxygen saturation, changes in body temperature, and changes in voice quality (hoarseness/vocal fatigue).

In some examples, the controller 50 may further include facial recognition and the occupants 20 may be uniquely identified using the images generated by the cameras 32, 34. The facial recognition technology automatically identifies which passengers are in the vehicle and adjust the sensitivity of the system in real-time to correlate with the profile data of the current occupants 20.

The vehicle 10 includes a heating ventilation and cooling (HVAC) system 40. The HVAC system 40 has an air intake 42 and an air outlet 44. In the example, the HVAC system 40, the air intake 42 draws air from both inside the passenger compartment 14 and outside the vehicle 10. The controller 50 is in communication with the HVAC system 40. Included within the HVAC system 40 is an air quality sensor suite 46. The air quality sensor suite 46 includes sensors for monitoring allergen quantities in the air provided to the passenger compartment 14, as well as the air ingested via the air intake 42.

The air quality sensor suite 46 monitors the airflow of the intake 42 and the outlet 44 for particles or substances that may be allergenic including common airborne allergens such as pollen, dust, mold, pet dander and the like, as well as monitoring for airborne traces of less common allergens including nuts, seafood, fruit/vegetables, latex, insects, medications and the like. In addition to allergen monitoring, the air quality sensor suite 46 monitors for air pollution, smog, industrial emissions, smoke, chemical fumes and odors. In some examples, data generated by the air quality sensor suite 46 can be supplemented by weather data generated by vehicle sensors including heat and cold extremes, humidity levels, and the like, as well as weather data provided to controller 50 from an exterior computer system 70 through a data connection.

Environmental factors can lead to longer and more concentrated pollen seasons. As a result, allergies are more common and people's reactions when allergies are triggered can be more severe. The vehicle 10 includes a non-invasive in-vehicle system (cameras 32, 34, air quality sensors 46 and physiological sensors 22 and allergen sensitivity module 52) that tracks changes in the sensitivity of one or more occupants 20 to allergens over time by analyzing air particulates and allergen concentration data collected via the air quality sensor suite 46 and correlating the collected data with physiological responses of the occupants 20 while in the vehicle 10. The physiological responses are identified by a combination of readings from the physiological sensor suite 22 and image analyses of video feeds provided by the cameras 32, 34.

The allergen sensitivity tracking takes advantage of the fact that the vehicle 10 is a contained system with routine activities (e.g., daily commutes, weekly activity trips, and the like). In addition to being a contained system, the vehicle 10 has a reduced number variables relative to a less constrained system, such as a building, due to the regular usage patterns. The reduced number of variables combined with being a contained system allows the controller 50 to more easily identify potential triggers for allergens, and the severity of those triggers for any given regular occupant 20. By analyzing the correlations in data, the vehicle 10 can provide insights into the air quality within the passenger compartment 14 and insights about potential allergen sensitivity changes in one or more of the regular occupants 20 of the vehicle 10. In some examples, the process 200 recommends that an occupant seek medical treatment and/or redirects a vehicle navigation system or an autonomous driving system to a treatment facility.

When a physiological response to an allergen is identified using the physiological sensor suite 22, the controller 50 notifies the occupant 20 what airborne matter is correlated with the response, allowing the occupant 20 to identify what triggered the response. In some examples, the controller 50 can also generate a report that can be shared with a medical provider. When the physiological sensor suite 22 detects signs of respiratory distress based on the data collected by the air quality sensor suite 46, the controller 50 can automatically intervene using pre-programmed preferences. The pre-programmed preferences can include but, are not limited to, adjusting in-cabin 14 environmental systems (air intake 42, air outlet 44, recirculation, and the like). In some examples, the controller 50 can further respond by providing guided breathing exercises, contacting an emergency contact, engaging an automated vehicle system to stop the vehicle 10 in a safe location, alerting a first responder and providing a stored medical history with the first responder, or rerouting the vehicle 10 through a less polluted area. In some other examples, the controller 50 may further identify a physiological response and provide notifications to the occupant 20. The notifications can include recommendations to visit a medical provider, notifications to a change in severity of the occupants reaction, or any similar notification.

In addition, in some examples, the controller 50 may crowd source data collection and correlate external conditions (e.g., vehicle settings, specific make/model/years of the vehicle 10, trim option, and other variables related to the effectiveness of filters, purifiers, etc.) using communication with the remote computer system 70 using any conventional communication process. This communication can further be utilized by the remote computer system to prevent sensitive drivers from entering areas where high levels of allergens could cause them issues.

With continued reference to FIG. 1, FIG. 2 illustrates a process 200 for monitoring allergens in the air and correlating the monitored allergens with physiological responses of occupants, and for using the correlation to track changes in an allergen sensitivity of an occupant 20.

Initially, upon startup of the vehicle 10, at a drive initiated step 202, the controller 50 recognizes the occupants 20 and integrates stored occupant profiles corresponding with the identified occupants 20 with the allergen sensitivity monitoring module 52 in an identification step 204. The occupants 20 may be recognized via facial recognition using cameras 32, 34, manual entries by the occupants, or any conventional identification manner.

As the vehicle 10 is operated, the air quality sensor suite 46 collects air quality and allergen information using the air quality sensor suite 46 and the controller 50 monitors the cameras 32, 34 and the physiological sensor suite 22 outputs to determine the physiological responses of the occupants 20 in a data gathering step 206. In addition to the monitored data, one or more occupants 20 may manually enter data in a self-reported data step 208. The manually entered data may be entered via a connected phone application, an in vehicle interface, or any other methodology for entering data.

As the vehicle 10 operates, the process 200 loops, beginning with an allergen sensitivity correlation calculation step 210. The calculated allergen sensitivity correlation defines the severity of physiological response vs the potency of the allergens (e.g., the parts per million of the allergen detected in the air). The correlations are evaluated against thresholds, with the thresholds being determined by those skilled in the art based on understandings of phsyiological responses to allergens. The severity of the physiological response is scored on a scale (e.g. 0 to 10, with 10 being most severe) and the potency of the correlated allergen is normalized to the same scale.

For the physiological response, the physiological response is measured by sensors, cameras, etc. and a severity is assigned such that a severity of 0.0 indicates no physiological response, and a severity of 10.0 is a maximal physiological response (e.g. anaphylactic shock).

Similarly, the potency of the allergen is normalized on a linear scale based on particulate matter with a 0 indicating that no allergen of that type is present, and a 10 indicating that an extremely high magnitude of that allergen type is detected. The allergen score is fit to the type of allergen by one of skill in the art, with the normalization being based off of conventional understandings of low, medium, high, and extreme allergen counts for a given allergen. The 0 to 10 range listed for the scores is an arbitrary range, and practical implementations may use any numerical scale where the range indicates a low to a high severity.

Using the scores, the process 200 determine if there is a correlation between the presence of any given allergen and a physiological response in an allergen correlation check 212. A correlation is identified when both the physiological response, and the potency have scores on their respective scales above a given threshold. When a correlation is identified, the process 200 generates an assessment report 214 and alerts the occupant(s) 20 to the correlation, as well as any other designated contacts that may be assigned by the occupant 20, in an alert occupant step 216. The process then returns to the data collection step 206 and continues iterating.

When the correlation check 212 does not identify a correlation, the process 200 allows the occupant(s) 20 to provide any additional symptoms that may not have been identified by the physiological sensor suite 22 in a confirm additional symptoms step 213. When the occupant(s) 20 confirm that additional undetected physiological symptoms were present, the additional symptoms are incorporated into the data set, the physiological response score is recalculated, and a second allergen correlation check 215 is performed. When a correlation is present at the second check 215, the process 200 generates the assessment at step 214 and proceeds from step 214.

When the additional symptom information does not result in an identified correlation at the second check 215, the process 200 reviews historical occupant data and determines if the allergen correlation of that particular occupant 20 has changed from previous assessments in a correlation changed check 218. In one example, this comparison is performed by identifying when the potency has been at similar levels and determining if the physiological response is different. If there has not been a change, the process 200 returns to the data collection step 206 and iterates. When the allergen correlation has changed, the process 200 generates a report in the generate report step 214 and proceeds from there.

While illustrated as a continuous iteration, it is appreciated that the process 200 can include a delay between each iteration, allowing for additional data to be collected before attempting to identify new or changed correlations. By way of example, the process 200 may collect data (data collection step 206) continuously, but only check correlations at the end of each trip, or every set time period (e.g., every minute).

In-vehicle allergy monitoring offers a controlled environment with real-time allergen data and physiological response tracking. This allows for continuous exposure tracking and personalized allergy profiles, potentially leading to earlier allergy identification and improved well-being for drivers.

The terms “a” and “an” do not denote a limitation of quantity, but rather denote the presence of at least one of the referenced item. The term “or” means “and/or” unless clearly indicated otherwise by context. Reference throughout the specification to “an aspect”, means that a particular element (e.g., feature, structure, step, or characteristic) described in connection with the aspect is included in at least one aspect described herein, and may or may not be present in other aspects. In addition, it is to be understood that the described elements may be combined in any suitable manner in the various aspects.

When an element such as a layer, film, region, or substrate is referred to as being “on” another element, it can be directly on the other element or intervening elements may also be present. In contrast, when an element is referred to as being “directly on” another element, there are no intervening elements present.

Unless specified to the contrary herein, all test standards are the most recent standard in effect as of the filing date of this application, or, if priority is claimed, the filing date of the earliest priority application in which the test standard appears.

Unless defined otherwise, technical and scientific terms used herein have the same meaning as is commonly understood by one of skill in the art to which this disclosure belongs.

While the above disclosure has been described with reference to exemplary embodiments, it will be understood by those skilled in the art that various changes may be made and equivalents may be substituted for elements thereof without departing from its scope. In addition, many modifications may be made to adapt a particular situation or material to the teachings of the disclosure without departing from the essential scope thereof. Therefore, it is intended that the present disclosure not be limited to the particular embodiments disclosed, but will include all embodiments falling within the scope thereof.

What is claimed is:

1. A vehicle comprising:
a controller having a processor and a memory, the controller including an allergen sensitivity detection module;
a physiological sensor suite configured to identify a physiological response of an occupant;
an air quality sensor suite including air quality sensors;
at least one camera defining a field of view including the occupant; and
wherein the allergen sensitivity detection module includes instructions configured to cause the processor to detect a physiological response of the occupant, identify a correlation between the physiological response of the occupant and a detected allergen, and generate a response to the identified correlation.

2. The vehicle of claim 1, wherein the response generated includes at least one of outputting an allergen sensitivity report to the occupant, notifying a first responder, adjusting at least one in vehicle airflow parameter, rerouting a vehicle navigation system, and rerouting an autonomous driving system.

3. The vehicle of claim 1, wherein the response generated is dependent on a severity of the physiological response.

4. The vehicle of claim 1, wherein the identifying the correlation between the physiological response of the occupant and the detected allergen comprises scoring a severity of the physiological response on a physiological response scale, normalizing a detected magnitude of at least one allergen to the physiological response scale, and identifying a correlation between the physiological response and the at least one allergen in response to a physiological response score exceeding a threshold and the normalized magnitude of the at least one allergen exceed the threshold.

5. The vehicle of claim 4, wherein identifying the correlation between the physiological response of the occupant and the detected allergen includes receiving at least one occupant entered physiological response and recalculating the physiological response including the occupant entered physiological response.

6. The vehicle of claim 1, wherein the air quality sensor suite includes a first set of sensors configured to monitoring allergen quantities in air ingested to the vehicle from an exterior environment and a second set of sensors configured to monitor air recirculated from a passenger compartment of the vehicle.

7. The vehicle of claim 1, wherein the allergen sensitivity detection module further includes instructions configured to cause the processor to update an occupant history file with the identified correlation and compare the identified correlation with historical correlations.

8. The vehicle of claim 7, wherein the generated response includes an output identifying a difference between the identified correlation and the historical correlations.

9. The vehicle of claim 1, wherein the physiological sensor suite includes biosensors, cameras, microphones, and lidar sensors.

10. The vehicle of claim 1, wherein the allergen sensitivity detection module includes instructions configured to cause the processor to iterate identifying the correlation between the physiological response of the occupant and a detected allergen, generate a response to the identified correlation at a predetermined interval.

11. The vehicle of claim 10, wherein the allergen sensitivity detection module includes instructions configured to cause the processor to continuously detect a physiological response of the occupant and monitor allergen levels.

12. A method for monitoring an allergen sensitivity of a vehicle occupant comprising:
detecting a physiological response of the occupant using a physiological sensor suite of the vehicle;
detecting a magnitude of at least one allergen in the vehicle using an air quality sensor suite of the vehicle;
identifying a correlation between the physiological response of the occupant and the detected magnitude of the at least one allergen using a controller of the vehicle; and
implementing a vehicle response to the identified correlation using the controller.

13. The method of claim 12, wherein the vehicle response includes at least one of outputting an allergen sensitivity report to the occupant, notifying a first responder, adjusting at least one in vehicle airflow parameter, rerouting a vehicle navigation system, and rerouting an autonomous driving system.

14. The method of claim 12, wherein the vehicle response is dependent on a severity of the physiological response.

15. The method of claim 12, wherein identifying the correlation between the physiological response of the occupant and the detected allergen comprises scoring a severity of the physiological response on a physiological response scale, normalizing a detected magnitude of at least one allergen to the physiological response scale, and identifying a correlation between the physiological response and the at least one allergen in response to a physiological response score exceeding a threshold and the normalized magnitude of the at least one allergen exceed the threshold.

16. The method of claim 15, wherein identifying the correlation between the physiological response of the occupant and the detected allergen includes receiving at least one occupant entered physiological response and recalculating the physiological response including the occupant entered physiological response.

17. The method of claim 12, wherein the air quality sensor suite includes a first set of sensors configured to monitoring allergen quantities in air ingested to the vehicle from an exterior environment and a second set of sensors configured to monitor air recirculated from a passenger compartment of the vehicle.

18. The method of claim 12, further comprising updating an occupant history file with the identified correlation and compare the identified correlation with historical correlations.

19. The method of claim 18, wherein the vehicle response includes an output identifying a difference between the identified correlation and the historical correlation.

20. The method of claim 12, further comprising iterating identifying the correlation between the physiological response of the occupant and a detected allergen, generate a response to the identified correlation at a predetermined interval.
